(19) United States
(12) Patent Application Publication
(10) Pub. No.: US 2008/0124781 A1
(43) Pub. Date: May 29, 2008

(54) Enzymes with modified amino acids

(76) Inventors: Rainer Bischoff, Groningen (NL); Robert Freije, Groningen (NL)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/977,839

(22) Filed: Oct. 26, 2007

(30) Foreign Application Priority Data
Apr. 26, 2005 (EP) PCT/EP04/51866

(51) Int. Cl. C12N 9/99; C12M 1/00; C12M 1/40

(52) U.S. Cl. 435/184; 435/283.1; 435/287.1

(57) ABSTRACT

An enzyme for protein digestion is provided, having at least one amino acid containing an N-terminal amino group and/or an amino group side chain, which is modified by a substituent introduced into the enzyme so as to reduce autodigestion and/or enhance protein digestion of the. Furthermore, a method for modification and immobilization of said enzyme is provided.

### BACKGROUND ART

The present invention relates to chemical modification of immobilized enzymes.

Enzymatic cleavage of proteins is an essential step in structure elucidation of an individual protein or of proteins in a mixture [Aebersold, R.; Mann, M. Nature 2003, 422, 198-207]. The peptides obtained by such cleavage reactions are easily transferred to a mass spectrometer allowing compositional analysis of individual peptides based on their exact molecular mass or sequence analysis by subsequent fragmentation in this instrument. The stepwise loss of an amino acid from the peptide chain permits to determine part of its amino acid sequence and therewith identify the protein from which it originates by comparative data base analysis. Alternatively, in case the protein is unknown in the data base, the protein can be partially reconstructed from the identified peptides and its cDNA cloned based on the partial sequence information.

The cleavage of proteins into peptide fragments amenable to mass spectrometry therefore is important, since proteins themselves are much more difficult to analyze and identify by mass spectrometry.

Enzymatic protein cleavage is brought about by proteases, protein cleaving enzymes. Several of such proteases are known with trypsin as the most well-known representative widely used in protein analysis. Such proteases cleave the protein at very specific locations specified by a composing amino acid. Trypsin cleaves specifically after (C-terminally) a lysine (K) or an arginine (R) in the peptide chain unless followed by the secondary amino acid proline (P). This type of cleavage is called digestion. Protein cleavage with trypsin is called trypsin digestion and the resulting peptide mixture a tryptic digest.

Trypsin digestion typically is carried out in a homogeneous solution of the enzyme with the protein or protein mixture. The ratio of trypsin to protein(s) is kept very low, (e.g. 1:100) since otherwise, products from self-digestion of the enzyme are found in the resulting peptide mixture. The reaction is often executed in a solution that denatures the protein(s) so that the locations for cleavage become readily accessible. The reaction in solution takes place at slightly elevated temperature (e.g. 35° C.) and requires 6-16 hrs. In most cases, the proteins are chemically treated prior to digestion to reduce disulfide bridges and to block the resulting thiol groups through alkylation.

In a protein mixture, like a proteome of particular cells or of a body fluid, the concentration of proteins present may have a range of 6 to 12 orders of magnitude. Consequently, because of the Michaelis-Menten kinetics, proteins present at very low concentration will digest slower than those present at high concentration. In proteomics though, the proteins with lowest concentration are often most interesting.

Speed and efficacy of the digestion process can be increased by immobilization of the proteolytic enzymes on a solid-support. Immobilization of trypsin on different solid-supports is reported by Canarelli et al (2002).¹ By immobilization, autodigestion is minimized and the speed of cleavage increases since the enzyme/substrate ratio at the support surface will be much more favorable.

Protein digestion with immobilized trypsin is carried out with flow-through devices like packed bed reactors or columns sometimes provided as a cartridge. Reaction time is governed by the flow rate and the volume of the device and ranges in practice from 1-20 minutes.

The dwindling size of samples in proteomics, whilst maintaining or even increasing the demand for high sensitivity, has led to miniaturization of reactors for on-line digestion in conjunction with miniaturization of the separation column. The use of immobilized trypsin in the field of microfluidics is referred to by Svec and Peterson et al., who have produced monoliths with immobilized trypsin, molded in both fused silica capillaries and microfluidic channels. Another approach is the use of membrane adsorbed trypsin reactors as reported by Gao (2003) and Cooper (2003, 2004).

Wang et al. (2000) describe on-chip digestion of trypsin immobilized on agarose beads followed by a separation in a glass CE chip before ESI/MS.

Slysz (2003) reports improved digestion efficiency of proteins with high proteolytical stability by using organic solvents in the digestion.

Keil-Dlouha (1971) found a reduced digestion efficiency and cleavage specificity, due to trypsin autolysis. An attempt to reduce immobilized trypsin autolysis by reductive methylation has been described by Davis (1995), however, at the expense of a decrease in overall proteolytic activity.

### DISCLOSURE

It is an objective of the invention to provide an improved protein digestion. The objective is solved by the independent claims. Preferred embodiments are shown by the dependent claims.

According to embodiments of the present invention enzymes for protein digestion are provided, having one or a plurality of amino groups or side chains containing amino groups, whereby an enhanced stability of the enzyme can be achieved due to modification of the amino groups, whether or not on side chains, and whereby a significant increase of the digestion speed and a reduced autodigestion of the enzyme is obtained.

One preferred embodiment refers to a modification of the enzyme trypsin, wherein the modification is obtained by acetylating the amine group of the amino acid side chains, thus acetylated groups are introduced into the enzyme.

A number of further embodiments refer to the enzyme being immobilized on a solid-support. Preferable supports comprise Sepharose, Agarose, polystyrene/divinylbenzene, silica and the like.

Another preferred embodiment refers to a cartridge for digestion of proteins comprising an enzyme according to an embodiment of the present invention, the enzyme being immobilized on solid-supports.

In a still further embodiment, said cartridge is comprised in a reactor for digestion of proteins, which reactor can be integrated in an automated protein analysis platform using on-line digestion.

Finally, in embodiments of the present invention the process of chemical modification of functional groups in enzymes and the immobilization process are described exemplarily, taking into account that the modification and immobilization process of numerous enzymes can be performed in analogy to the process described for the modification and immobilization of trypsin.

### BRIEF DESCRIPTION OF FIGURES

FIG. 1 shows the results of HPLC analysis of trypsin immobilization on Sepharose beads. The immobilization supernatant before and after immobilization is depicted in absence and in presence of benzamidine.

FIGS. 2a and b show the effect of the modification (2b) on the trypsin digestion efficiency of 4 μM cytochrome c, analyzed by LC-MS in comparison to regular Trypsin (2a); Trypsin being immobilized on different solid-supports; (A) Sepharose, (B) Agarose (Pierce beads) and (C) Poroszyme®.

FIG. 3 shows the effect of acetylation on autolysis peptides from trypsin, immobilized on Sepharose, with the upper trace being obtained using a regular trypsin cartridge, and the lower trace representing a modified trypsin cartridge.

FIG. 4 (table 1) shows identified trypsin autolysis peptides with the peak numbers corresponding to the immobilized trypsin autolysis peptides as presented in FIG. 3.

FIG. 5 shows the LC-MS analysis of the cytochrome C digestion kinetics with differentially acetylated, soluble trypsin.

FIG. 6 gives the correlation between catalytic efficacy (k_{cat}/Kₘ) and the degree of soluble trypsin acetylation determined with Z-FR-AMC (▪) and Z-LR-AMC (♦).

FIG. 7 (table 2) shows the determination of the biochemical rate constants for the conversion of two fluorescent substrates upon acetylation of soluble trypsin to varying degrees.

### DESCRIPTION

Many of the attendant advantages of embodiments of the present invention will be readily appreciated and become better understood by reference to the following more detailed description of embodiments.

Before the embodiments of the invention are described in detail; it is to be understood that this invention is not limited to the particular compounds such as solid-supports like Agarose or Sepharose and is not limited to process steps of the methods described, as such chemical compounds may be substituted and methods may vary. It is also to be understood, that the terminology used herein is for purposes describing particular embodiments only and it is not intended to be limiting. It must be noted that, as used in the specification and the appended claims, the singular forms of “a”, “an”, and “the” include plural referents until the context clearly dictates otherwise.

Generally, embodiments of the present invention aim for an improved stability of protease and provide an enhanced digestion of proteins by modification of the protein digesting enzyme or protease, respectively. The modification is obtained by a chemical reaction of the immobilized enzyme, which can be one of Trypsin, Pepsin, Lys-C, Glu-C, Chymotrypsin, Arg-C, Asp-N, elastase or Papain with a modifying reagent, suitable to provide “blocking” of the amino group or said side chain amino group comprised of the amino acid or acids, respectively.

“Blocking” means herein to couple the N-terminal amino acid and/or amino-group containing amino acid to another molecule in a manner that autodigestion of the enzyme becomes inhibited or diminished and enzymatic activity may be enhanced. Due to that modification stabilization of the enzyme is achieved.

Blocking of the enzyme Trypsin can be achieved by introducing an acetyl group into the molecule. This is achieved by performing an acetylation of the trypsin N-terminus and the amino group of the amino acid lysine. The accompanying figures indicate clearly that acetylation of immobilized trypsin results in an enhanced activity. Of course the effect described herein can be achieved by other functional groups, leading to an enhanced activity and/or stability of the protease, too.

Furthermore functional groups resulting from the reaction with ethylene glycol bis-(succinimide succinate) or conjugations with sugars can be useful to achieve blocking in the above sense. One can as well perform a reaction with polyethylene glycols (PEGs). PEGs with different lengths can be taken; one may chose for example methoxy-polyethylene glycol which is activated by N-hydroxy-succinimide (NHS) or which is alternatively activated with p-nitrophenyl chloroformate (NPC) or related activated carbonates familiar to one skilled in the art of protein modification.

Another option is to couple carboxylic acid anhydrides to the primary amine groups of lysine thereby reversing the positive into a negative charge thus modifying the surface properties of the enzymes. Reaction with succinic acid anhydride is an example of such a reaction.

Further options are conjugation with acid chlorides, mixed anhydrides or activated carboxylic acid esters leading to amides of the corresponding amine groups in the enzymes with the organic functional groups R of the carboxylic acid as residues. Details about reaction conditions are well known to the person skilled in the art.

In order to carry out protein digestion, Trypsin is advantageously immobilized on a suitable solid-support material. Sepharose can serve as solid-support, in particular N-hydroxysuccinimide activated Sepharose as used for the tests that are outlined in the accompanying figures. Advantageously, the Sepharose material is provided as beads, thus offering a preferred surface.

The accompanying figures refer to results of experiments carried out with Sepharose, Agarose (Pierce beads) and polystyrene/divinylbenzene (Poroszyme®) as solid-supports. Further solid-supports can be provided by other polystyrene-based solid-supports or alternatively, one can chose silica- or nitrocellulose-based solid-supports as well as materials containing a paramagnetic core (e.g. Dynabeads®) for sample handling in robotic devices.

It has to be taken into consideration that the solid-support material must not necessarily be bead-shaped, other forms are alternatively possible. In particular, utilization of monolithic solid-supports, membranes or planar solid supports, such as microfluidic channels and the like, can be advantageous.

A further aspect of embodiments of the present invention is the integration of immobilized and acetylated trypsin beads for enhanced digestion efficacy in reactors: Herewith one can obtain digestion reactors with higher activity—with respect to the ones known in the state of the art—in order to make a broader part of the proteome accessible to peptide mapping. Since low digestion yields at low protein levels and band broadening of peptides on protease reactors are obstacles for on-line digestion of low abundance proteins, an enhanced digestion efficiency resulting in increased digestion yields would enhance the mapping process. It has to be taken into consideration that one may wish to apply the technology described herein on devices having a size which ranges from the conventional lab reactor down to micro sized reactors such as microfluidic devices.

Because of the increased trypsin activity, digestion reactors can be scaled down, which results in a reduction of peptide losses caused by non-specific binding and band broadening. Hence, on-line digestions with low abundance proteins can be performed with higher yields, thus making the use of chemically modified immobilized trypsin beads a valuable tool in automated protein analysis systems. Said scaled-down reactors can be utilized in an automated protein analysis platform using integrated on-line digestion before separation, for example based on reversed phase chromatography, is carried out. Thus the need to perform analyses on increasingly limited quantities of proteins can be accommodated.

It must be noted that the above modification and immobilization being described exemplarily with trypsin can also be performed with other proteases such as pepsin, Lys-C, Glu-C, chymotrypsin, elastase, Arg-C, Asp-N, elastase or papain, which also undergo a stabilization and reduction of autodigestion when being modified, and, hence, show a clearly defined cleavage specificity then, which increases the reliability of analysis results and facilitates the interpretation of the peptide maps.

### General Method of Immobilizing and Modifying Trypsin, Pepsin, Lys-C, Chymotrypsin, Glu-C, Arg-C, Asp-N, Papain, or Elastase:

Generally, immobilization of the selected protease is carried out first, before a mild modification is performed.

The following reagents can be used:

### Wash buffer 1, wash buffer 2, a coupling buffer, a modification buffer comprising the modifying reagent and a blocking buffer.

In order to immobilize the desired protease on a desired solid-support, said solid-support material can be subjected to preparatory steps, whereby impurities can be removed from the solid-support material and whereby the solid-support material is brought to the optimal pH value with respect to the enzyme, the chosen immobilization chemistry and with respect to the solid-support material. Sepharose, in particular an N-hydroxysuccinimide activated Sepharose, agarose, polystyrene/divinylbenzene-based solid-supports such as Poroszyme®, Nitrocellulose, Dynabeads® or silica-materials can serve as solid-support. The material can be formed like beads or it can be monolithically shaped or be in the form of a membrane or a planar surface such as microfluidic channels.

A first preparatory step is the washing of the solid-support material with the washing buffers 1 or 2, or both of them, if necessary. One should perform the washing process at a suitable temperature at about 2 to 8° C. with an appropriate volume of wash buffers. Of course, said preparatory step may vary as well, depending on the used immobilization chemistry and stability of reactive groups.

The needed volume of enzyme is dissolved at 0° C. in coupling buffer, then one can add it to the solid-support and incubate it for a predetermined time of about 25 min. at a temperature of 25° C., depending on the chosen chemistry, while it is subjected to rotary shaking (1100 rpm gives an indication).

Temperature and time of incubation may vary; furthermore one can chose another method than rotary shaking to provide optimal mixing of enzyme and solid-support, depending on the embodiment of the solid-support. Whereas beads-shaped solid-support materials can be rotary shook, one may chose a vibrator for preparation of a monolithic material or perform the modification in a flow-through system.

After immobilization the supernatant liquid can be removed and the immobilized enzyme can be modified by the addition of an equal volume of modification buffer. The modification buffer contains the modification reagent, which can be

an acetyl group providing reagent such acetic acid N-hydroxy-succinimide ester (AANHS)

or a reagent providing cross links such as ethylene glycol bis(succinimidyl succinate)

or a sugar conjugate providing reagent such as a reagent comprising monomeric or oligomeric sugars like cyclodextrine, whose functional hydroxyl-groups were converted into aldehydes, for example through oxidation with sodium periodate,

or a succinyl-group providing reagent,

a guanidyl-group providing reagent,

or a nitrosyl-group providing reagent, which after reduction would allow to introduce aromatic amino groups into tyrosine for subsequent modification,

conjugation with glyceraldehyde.

Said reagents provide introduction of the desired residue or functional group, respectively, in the enzyme by coupling with the amino-group comprised by the relevant amino acid. It has to be taken into consideration that also modification of side chains, such as e.g. tyrosine side chains, are comprised by embodiments of the present invention.

So, the amino group of interest can be part of the terminal amino acid or any one of the alpha-, beta- or higher standing amino acids. The concentration of the modification reagents determines the number of amino group containing amino acids to react, hence partial or complete modification is achieved. The modification reagent reacts with the immobilized enzyme for an incubation time of approximately 20 minutes. Of course, one may choose a longer incubation time. The incubation temperature can be 25° C. while rotary shaking at about 1100 rpm is performed. Of course, the reaction conditions may vary.

Excess modification reagent can be blocked by addition of 5 volumes of blocking buffer; the blocking reaction is carried out during 10 minutes of incubation at 25° C. while rotary shaking at about 1100 rpm is performed. The blocking reaction conditions may vary, too. The reaction time may range from 1 min to 4 hours; the temperature range is from 0° C. to 60° C., depending on the stability of the enzyme.

Immobilization of proteolytic enzymes on monolithic materials can be achieved by flowing the various buffers and activating solutions through a capillary, cartridge or microfluidics device in the order described for batch immobilization on beads. An additional possibility with monoliths is to immobilize the enzymes through entrapment in the monolith itself during the sol-gel reaction. Such entrapment may also be effected during the synthesis of particulate materials such as beads.

It can be desirable to perform digestion of proteins with enzymes according to embodiments of the present invention by utilization of cartridges. Generally, the immobilized enzyme is brought into a cartridge then. When the solid-support material comprising the enzymes is beads shaped, a storage buffer can be added to the beads and the resulting slurry is poured into the cartridge. A monolithic solid-support can be incorporated in an encasement, thus forming another type of cartridge. Membrane supports may also be used in the form of cartridges.

The cartridge can be integrated in a digestion reactor, additionally comprising devices such as sample inlets and outlets, comprising valves probably, or coupling means to transfer the digested proteins directly into analytical devices such as HPLC, MS or other devices suitable for protein mapping.

Finally, the reactor using a modified immobilized protease according to an embodiment of the present invention can be integrated in a multidimensional automated proteomics platform in order to allow the analysis of a broader range of the proteome with a higher dynamic range due to the integrated on-line digestion. Additionally, such an approach may reduce material losses of proteins and fragments, due to elimination of transfers and manipulation of diluted solutions of such protein fragments.

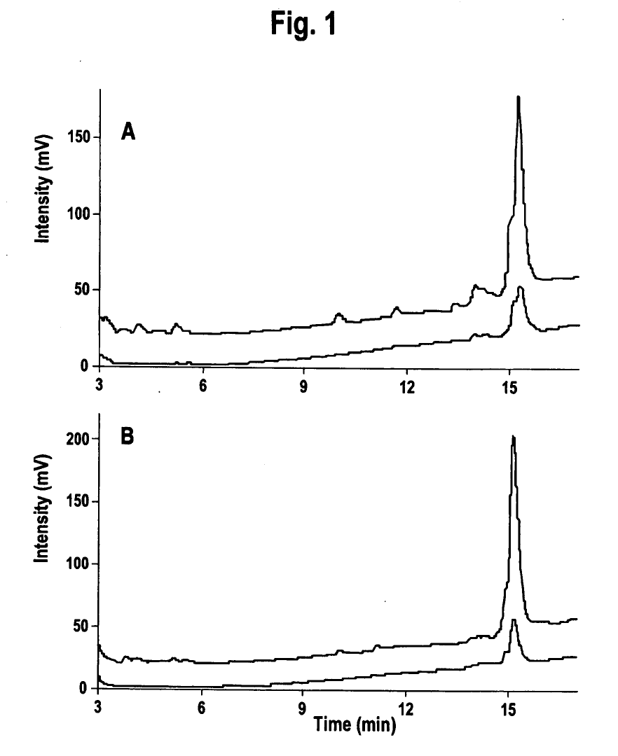

In the following, a number of experiments are given, describing the performance of the methods being embodiments of the present invention. The experiments are referred to by the FIGS. 1 to 7.

### Materials and Methods

### The Following Chemical Reagents and Materials have been Used for the Experiments Described Below:

Trypsin (TPCK treated, bovine pancreas), cytochrome c (bovine heart), benzamidine, calcium chloride, ethanolamine, trifluoroacetic acid and NaN₃ were purchased from Sigma, formic acid was obtained from Merck KGaA. AANHS, Brj-35 and tris(hydroxymethyl)aminomethane (Tris) were from ICN Biomedicals and NHS-activated Sepharose 4 fast flow was from Amersham. Acetonitrile was form Biosolve. Ultra-pure water was used for all buffer and mobile phase preparations.

### Trypsin Immobilization and Modification with NHS-Activated Sepharose, Agarose and Poroszyme®.

The below solutions are suggested to be used for trypsin immobilization:

wash buffer 1: 1 mM HCl;

wash buffer 2: 0.1 M K₂HPO₄, with a pH 7.8;

coupling buffer: 0.1 M K₂HPO₄, 5 mM ethanolamine, with or without 4 mM benzamidine with a pH 7.8;

modification buffer: 0.1 M K₂HPO₄ pH 7.8, 22 mM AANHS

blocking buffer 0.5 M ethanolamine with a pH 8.0.

A) Immobilization on NHS-Activated Sepharose Beads:

The NHS-activated Sepharose beads are washed at 4° C. with 10 volumes of washing buffer 1 and 2. An equal volume of 20 mg/ml trypsin, dissolved at 0° C. in coupling buffer, is added to the beads and incubated for 25 min. at 25° C. and rotary shaken at 1100 rpm. After immobilization, the supernatant is removed and trypsin beads become modified by the addition of an equal volume of modification buffer (20 min. incubation at 25° C. and 1100 rpm). Excess of reactive NHS groups are blocked by the addition of 5 volumes of blocking buffer (incubated for 10 min., as described before).

B) For Pierce trypsin beads, modification is performed after washing with wash buffer 2, then the modification procedure according to A) is carried out.

C) For Poroszyme® trypsin beads, modification is performed after washing with wash buffer 2, then the modification procedure is carried out according to A), too.

Of course other types of trypsin beads can be used. All the types of trypsin beads used in the examples A) to C) were stored at 4° C. in 50 mM Tris pH 8.2, 1 mM CaCl₂, 0.02% NaN₃. Storage can be done under different conditions.

### HPLC Analysis

Samples from the trypsin solution before and after immobilization according to the above method have been diluted 80 times with 0.1% TFA in water and analyzed with HPLC using Merck-Hitachi equipment on a Vydac C₈ column (250 mm, 2.1 mm i.d., 5 μm, 300 Å pore size), detection at 214 nm, 20 μl injection volume, mobile phase from 25% to 55% acetonitrile (in water+0.1% trifluoroacetic acid) in 25 min.

### Digestion Experiments

Different trypsin beads have been slurry-packed with storage buffer into cartridges. A preferred cartridge can measure 10 mm (length)×1 mm or 2 mm (internal diameter) comprising stainless steel frits with 2 μm pore size. The samples were pumped through the cartridge which is housed in a clamp by use of a syringe pump (KD Scientific). The cartridge holder and cartridges are produced by Spark-Holland (Emmen, The Netherlands).

Of course, the use of trypsin beads or the use of any enzyme immobilized on a solid-support according to an embodiment of the present invention is not limited to be used in cartridges of said size. Other cartridges can also be used.

The trypsin cartridges were washed with 20 cartridge volumes of 50 mM Tris having a pH 8.2, 50% acetonitrile, followed by 20 cartridge volumes of digestion buffer (50 mM Tris pH 8.2) before sample loading. Digestion of protein samples is performed at room temperature unless indicated otherwise. Cytochrome c digestion was performed at 4 μM with 1 mm cartridges at a flow rate of 40 μl/min (appr. contact time 4 sec).

Trypsin autolysis experiments were performed with 2 mm cartridges packed with trypsin immobilized on Sepharose. Directly after washing with 50 mM with Tris pH 8.2, 50% acetonitrile, 120 μl digestion buffer was pumped through the cartridges at 4 μl/min (appr. contact time 3 min). The flow through was collected and combined for LC-MS analysis.

### LC-MS Analysis

All protein digest analyses were performed on an Agilent 1100 capillary HPLC system coupled on-line to an SL ion trap (Agilent, Benelux) equipped with a Vydac C8 column (250 mm, 1 mm i.d., 5 μm, 300 Å pore size). For each run 16 pmol of total protein digest was injected. For the autolysis experiment, 8 μl was injected. Peptides were eluted in a linear gradient (0.75% acetonitrile/min) from 3 to 47% acetonitrile with 0.1% formic acid at a flow-rate of 65 μl/min.

### Results Referring to Studies Concerning Immobilized Enzymes on Sepharose (A), Agarose (Pierce Beads) (B) and Poroszyme® Beads (C):

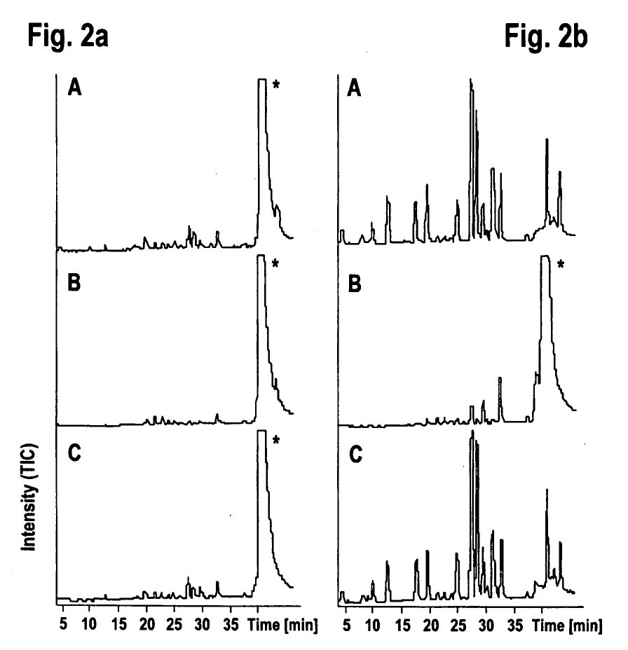

FIGS. 2a and 2b refer to studies concerning immobilized trypsin on the following different solid-supports: Sepharose (A), Agarose (Pierce beads) (B) and Poroszyme® beads (C). It can be seen clearly that the acetylation of immobilized trypsin (3 diagrams in FIG. 2b) leads to a striking enhancement of the Cytochrome C digestion activity in comparison to regular trypsin (3 diagrams in FIG. 2a). Intact Cytochrome C peaks are denoted by asterisks, pointing out the effect of the modification on the trypsin digestion efficiency of 4 μM Cytochrome C, analysed by LC-MS using a C8 Vydac column and an SL ion trap (Agilent). In FIG. 2b the large peak at the end of the gradient in the middle panel corresponds to undigested Cytochrome C.

The top panel results of FIGS. 2a and 2b have been obtained with Sepharose (A), the middle panel results with Agarose Pierces (B) and the bottom panel results with Poroszyme® (C). Notably, acetylation of the Poroszyme® trypsin cartridge with AANHS allowed to increase the digestion rate dramatically, which is an indication that the modification of enzymes leads to strongly enhanced digestion rates. It indicates further that the methodology of acetylating trypsin after immobilization is applicable to different kinds of proteolytic enzymes on different stationary phases.

FIGS. 2a and 2b show clearly that digestion is much faster with modified trypsin on all 3 stationary phases in comparison to the non-modified materials. Taking the two largest peaks of FIG. 2a—the middle—into account, e.g., the upper and bottom panels show an increase in peak height of about 5-10 fold in comparison to the corresponding peaks in FIG. 2b. FIGS. 2a and 2b indicate that there is an enhanced proteolytic activity for a protein substrate based on 3 different immobilized enzyme preparations.

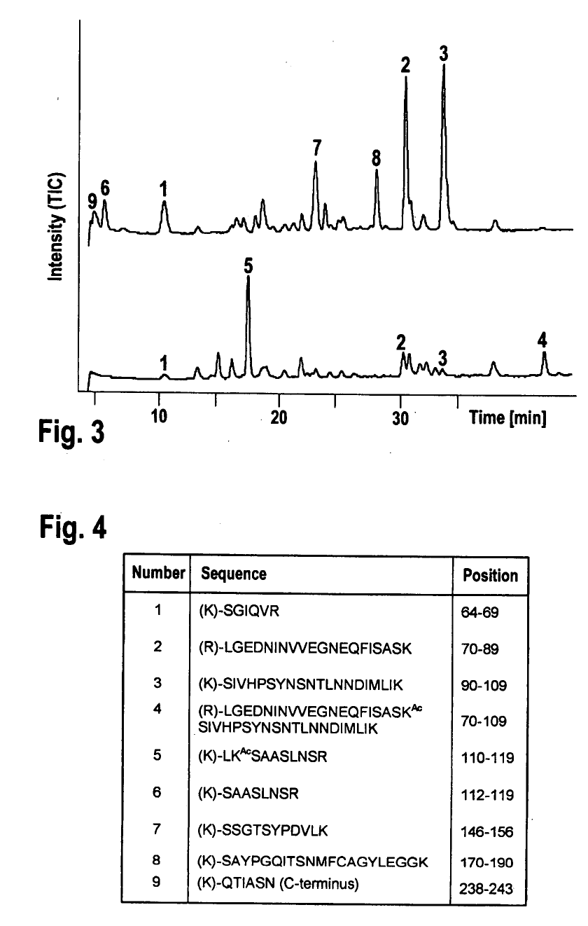

FIG. 3 describes that acetylated, immobilized trypsin is less prone to autodigestion than non-modified, immobilized trypsin. The table in FIG. 4 gives the identified trypsin autolysis peptides. The peak numbers correspond to the immobilized trypsin autolysis peptides as presented in FIG. 3. The amino-acid, preceding the scissile bond of the autolysis peptide is given between brackets. The position is indicated according to the sequence entry in the Swiss-Prot database (accession number P00760). While some autolysis peaks completely disappeared, others are strongly reduced but two new peptides appeared after modification.

The explanation for the appearance of peptides 4 and 5 can be found in FIG. 4. The mass increase corresponding to one acetylation, in combination with one missed cleavage after a lysine residue proves the acetylation of Lys 89 and Lys 111 in these two peptides. Although strongly reduced, autolysis peptides 2 and 3 are still present after acetylation, showing that acetylation of Lys 89 is not complete. Further information about the acetylation pattern can be derived from other changes in peptide signals, indicating that Lys 111, 145, 156, 169, 190 and 237 are also modified. From the appearance of peptide 4 and 5 one can conclude that Lys 109 was only little modified under the employed conditions.

To summarize, it could be said that there is less autodigestion, indicated by a lower amount of peptides generated (integrating the surface area could be used as a quantitative measure—see below). However, acetylated trypsin generates other peptides (see peptide number 5) that are not present in unmodified trypsin because they result from the modification itself, blocking certain cleavage sites. It can also be rationalized that acetylated trypsin has less internal cleavage sites, since most of the lysine residues are “blocked”. Cleavage after arginine is still ongoing.

Generally, one way of determining when autodigestion is reduced includes incubating the immobilized enzyme reactor in digestion buffer for extended periods of time (1 min to 48 h) at various temperatures (20-55 C) and collecting the buffer afterwards for LC-UV-MS analysis. The amount of peptides as a result of autodigestion can be quantified by integrating the area under the curve of the chromatogram obtained by UV (214 nm) or the Ion Current (total or extracted) obtained by mass spectrometry. Autodigestion may be reduced by varying the experimental conditions of the digestion but this will also have a concomitant negative effect on the digestion of other protein substrates, which is the goal in proteomics. Herein, however, autodigestion is reduced by modifying the enzyme itself thus reducing the number of accessible cleavage sites while even increasing enzymatic activity.

Referring again to FIG. 3, it can be seen clearly by scanning the lower panel that acetylated immobilized trypsin is significantly more stable towards autodigestion than the non modified immobilized trypsin, which is referred to in the upper panel. Immobilization was in both cases performed with Sepharose A. Analysis was performed by LC-MS using a C8 Vydac column and an SL ion trap (Agilent).

Furthermore, studies have been made with cartridges which have been used over 3 months with no obvious loss of activity. The cartridges were stored in digestion buffer at 4° C. The results are not figuratively shown herein.

The results shown in FIG. 1 emphasize the importance of the presence of a reversible enzyme inhibitor during the immobilization process: Herein, benzamidine was selected as said inhibitor, indicating that trypsin autodigestion can be reduced before immobilization, as judged from a reduced number of earlier eluting peaks before trypsin immobilization. The immobilization supernatant is given before (upper traces) and after (lower traces) immobilization, in absence (upper panel) and in presence (lower panel) of 4 mM benzamidine. The concentration of active-immobilized trypsin is most likely higher after immobilization with benzamidine because of reduced autolysis before and during immobilization.

### Method of Modifying Soluble Protease:

It can be desirable to modify soluble enzymes such as trypsin, pepsin, lys-C, chymotrypsin, glu-C, arg-C, asp-N, papain, or elastase without immobilizing them. One may wish to have such modified enzymes to perform kinetic measurements, e.g., in order to obtain information with respect to the digestion rate of a given protein that one is aiming for or for stability reasons (less autolysis).

### General Method:

A soluble enzyme can be modified by dissolving said enzyme in a suitable solvent or buffer, followed by stepwise adding the desired modification reagent to the solution containing the enzyme. Preferably, this is done in the presence of benzamidine to reduce autolysis. The pH of the reaction is slightly basic in the case of modifying primary amine groups. Temperature also needs to be adjusted dependent on the coupling chemistry but room temperature is preferred if possible. To optimize mixing of the reagents one can stir or mix by rotary shaking. The reaction is terminated when the samples are diluted to a predetermined volume of buffer. Adding of buffer is terminated when the desired pH-value and the desired enzyme concentration is obtained. The diluted samples can be stored on ice for further experiments. Proteolytic enzymes can be modified after immobilization or prior to immobilization. Then different types of modification chemistry and immobilization chemistry must be used: which means that side chains of different amino acids have to be used for modification and immobilization, otherwise all sides needed for immobilization are blocked by the modification.

The extent of modification and the level of heterogeneity during the modification reaction can be monitored by direct infusion-MS measurements. Samples are diluted with the infusion solvent to a definite enzyme concentration, followed by injecting them at a definite flow rate into the MS-device.

### Exemplary Differential Modification of Soluble Trypsin

Soluble trypsin was acetylated by stepwise addition of 1 M AANHS, which was dissolved in acetonitrile, to a 0.5 mM trypsin solution in 20 mM K₂HPO₄, and 5 mM benzamidine, having pH 8.0 at 25° C. It was rotary shaken at 900 rpm. The AANHS solution was added with increasing volumes at 0, 7, 14 and 21 minutes, resulting in freshly added concentrations of 5, 10, 15 and 20 mM respectively. The reaction was terminated by dilution of the samples, taken in time from the reaction mixture, with 50 mM Tris pH 8.5 until a final trypsin concentration of 1.25 μM was obtained. The diluted samples were stored on ice for further experiments.

The extent of modification and the level of heterogeneity during the modification reaction was monitored by direct infusion-MS (SL ion trap) measurements. Samples were diluted to 5 μM trypsin with the infusion solvent, acetonitrile/water 2:3 (v/v) and 0.1% formic acid, and were infused at a flow rate of 5 μl/min with a KD Scientific syringe pump.

### Kinetic Measurements Performed with Soluble Trypsin with Different Degrees of Modification

Soluble trypsin with different degrees of modification was used to determine the rates of Cytochrome C digestion. The digestion reaction was performed with 500 μg/ml Cytochrome C and 20 μg/ml trypsin in 50 mM Tris, pH 8.5 at 37° C. and 900 rpm (rotary shaking). The digestion reaction was monitored, after 10-fold dilution of the samples with 0.25% formic acid in water, by LC-MS analysis as described above.

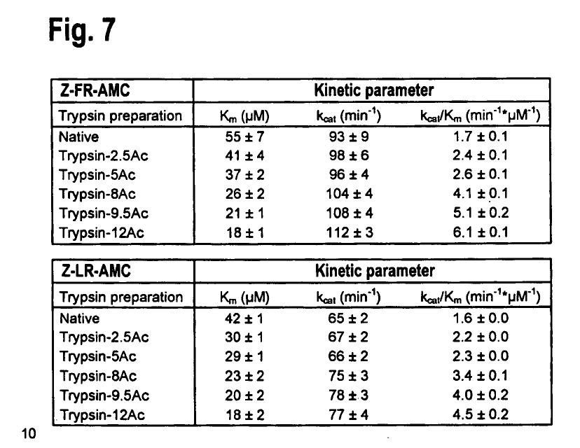

The enzymatic activity was determined by measuring the proteolysis rates of the profluorescent substrates Cbz-LR-AMC and Cbz-FR-AMC (Sigma) in duplicate at 12.5, 16.7, 25 and 50 μM, with 10 nM of differentially acetylated trypsin in a 50 mM Tris buffer (pH 8.5) containing 10 mM CaCl₂ and 0.01% Brij-35 (w/v). The assay was carried out at 25° C. in 96-well plates (Costar-white) and monitored over 4 min. with a Fluorostar optima plate reader (BMG Labtech) with λ_{ex, em}=390, 440 nm. Kₘ and k_{cat} values were obtained from Lineweaver-Burk plots (see FIG. 7). Measuring the enzyme activity in solution using a profluorescent substrate is an easy and quantitative way of showing that an increasing number of modifications of trypsin enhances its catalytic activity for two different low-molecular weight substrates, as the quantitative results given in FIG. 7 indicate.

### Results and Discussion

Trypsin has been immobilized in a one-step reaction to N-hydroxysuccinimide (NHS) activated Sepharose. Commercially available, pre-immobilized trypsin materials based on agarose and polystyrene/divinylbenzene (Poroszyme®) have also been used. As one example for embodiments of modifications according to the present invention the mild single-step lysine modification with AANHS has been outlined, stabilizing the enzyme against autolysis and because of the most advantageous effect of introducing minor steric changes.

It has been shown that the acetylation of immobilized trypsin leads to a striking enhancement of the Cytochrom C digestion activity, see FIGS. 2a and 2b. The active-site accessibility, a factor of major importance in protein digestion, especially for immobilized enzymes, which are already in close proximity of a solid-support, is not negatively affected by the introduction of the small acetyl group at lysine side chains.

Also noteworthy is the fact that the increased digestion efficiency is independent of the solid-support and nearly complete digests were obtained for modified Poroszyme® and Sepharose trypsin beads with a residence time of only 4 seconds of the substrate (Cytochrome C) in the enzyme reactor cartridge.

The effect of chemical modifications on soluble trypsin activity has thus far mainly been studied with low molecular weight substrates, generally resulting in a slight activity increase. The inhibited digestion of casein and blocked digestion of bovine serum albumin after trypsin conjugation with bulky β-cyclodextrins and an extremely bulky carbohydrate containing polymer indicates the importance of steric factors in protein digestion. Since acetylation causes only minor steric changes it is a valuable discovery that chemical modification does not have to result in reduced protein digestion rates.

For the determination of an optimized modified immobilized trypsin reactor the optimal acetylation degree for protein digestion has been studied with soluble trypsin, because the soluble acetyl-trypsin species are well characterized in terms of acetylation degree. Four different acetyl conjugated trypsin species were used to monitor the kinetics of in-solution digestion of Cytochrome C by LC-MS analysis. The digestion kinetics were determined by plotting the level of intact Cytochrome C and three fully digested peptides against the digestion time (as determined by peak area in extracted ion chromatograms).

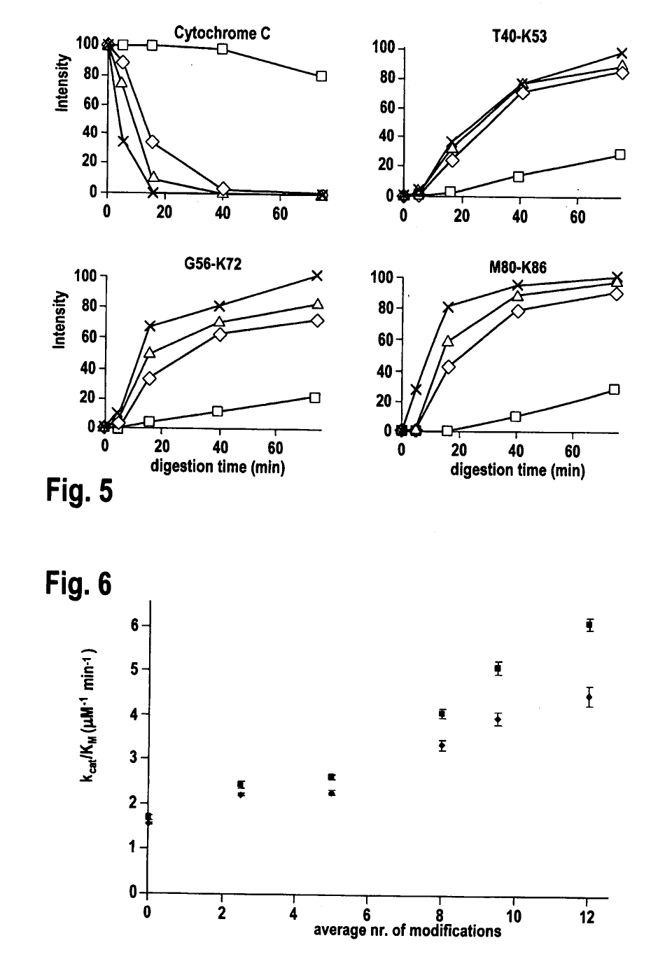

FIG. 5 (legend: Native trypsin (□) and trypsin with an average of 4, 7 and 11 acetyl conjugations, respectively (⋄, Δ, x) confirms that the modification-dependent increase in trypsin activity towards Cytochrome C digestion that we observed for immobilized trypsin is independent of chromatographic effects. While Cytochrome C has been completely degraded to fragments (regardless of the size) within 40 minutes for all three types of modified trypsin, almost 100% is left for native trypsin (an estimated 10-50 fold difference) and the digestion of Cytochrome C with native trypsin reaches only 20% after 75 minutes. Almost the same trend is observed for the three fully digested peptides (no missed cleavages), be it on a longer time scale because these are the end products in the digestion pathway. (Near) maximal digestion is reached in around 75 minutes with the three types of acetylated trypsin while the conversion with native trypsin remains around 20% on this time scale. With respect to the digestion kinetics, differences between different species of acetylated trypsin are less strong but still significant. Trypsin with the highest acetylation degree digests Cytochrome C with the highest rate. This likely translates also for immobilized trypsin, where the degree of acetylation is not easily determined.

The time point at 15 min shows clearly that the higher the degree of modification the faster the digestion rate (disappearance of undigested Cytochrome C).

To investigate how the enzymatic kinetic properties depend upon acetylation, the Michaelis-Menten kinetics of trypsin in-solution have been determined with the profluorescent substrates Cbz-Phe-Arg-AMC and Cbz-Leu-Arg-AMC.

FIG. 7 (Table 2) shows that the increased digestion efficiency towards Cytochrome C upon acetylation is reflected by lower Kₘ and higher k_{cat} values for the low molecular weight substrates. These changes in biochemical rate constants may also explain the increased overall activity of the Cytochrome C digestions with soluble and immobilized trypsin.

In general, enzymes with the ultimate combination of a high k_{cat} and low Kₘ are the most efficient biocatalysts especially at low substrate concentrations. Since the on-line digestion of low abundance proteins is still difficult, even with immobilized trypsin, the improved biochemical reaction constants of acetylated trypsin can contribute to higher digestion efficiency at low protein concentrations in proteomic methodologies as the protein concentrations will be less limiting in on-line peptide mass fingerprinting procedures with immobilized and chemically modified trypsin.

The efficiency of an immobilized enzyme reactor is largely determined by two intrinsic factors namely, the catalytic activity of the immobilized enzyme and the mass transfer rate of the substrate from the mobile to the stationary phase. This is illustrated by the fact that the most efficient proteolytic reactors reported thus far are based on membranes or monoliths, where the nearly complete lack of diffusion limitations can result in protein digestion in only a few seconds. With these solid supports, the main limitation for the digestion speed is the activity of the immobilized enzyme itself. With our post-immobilization-modification approach we have demonstrated that chemical modification can also contribute to the efficiency of immobilized enzyme reactors, as the limitations based on diffusion in porous beads are not altered upon modification.

Generally one can say that the modification of immobilized trypsin results in the ultimate combination of reduced autolysis and increased activity with respect to digestion. The increased activity can contribute to higher digestion yields of protein at low concentrations in samples in two ways. The dimensions of the digestion reactor can be scaled down while maintaining the overall digestion efficiency because of the higher activity. This leads to a reduced non-specific binding of protein substrates or tryptic peptides to the solid support. The effect of the modification (e.g. acetylation) may be based on a reduction of ionic interactions. Hence, the digestion can be done with higher yield and less dilution. Furthermore the digestion speed can be increased at a given solid-support concentration due to the higher trypsin activity. This makes the digestion of low concentration protein samples faster and hence lower amounts of protein can be digested with higher yields. Another advantage of higher digestion yields at low protein levels is the reduction in the need for post digestion concentrating steps which makes automated systems more complicated than desirable. Consequently, a broader range of the proteome can be measured with a higher dynamic range by using a modified immobilized trypsin reactor in a multidimensional automated proteomics platform.

### REFERENCES

Aebersold, R.; Mann, M. Nature 2003, 422, 198-207
Massolini, G.; Calleri, E. J Sep. Sci 2005, 28, 7-21
S. Canarelli, I. Fisch, R. Freitag, Hyphenation of multi-dimensional chromatography and mass spectrometry for the at-line-analysis of the integrity of recombinant protein drugs, J. Chrom. B 775 (2002) 27-35V,
J. W. Cooper, J. Chen, Y. Li, C. S. Lee, Membrane based nanoscale proteolytic reactor enabling protein digestion, peptide separation, and protein identification using mass spectrometry, Anal. Chem. 71 (2003) 1067-1074J,
M. T. Davis, T. D. Lee, M. Ronk, S. A. Hefta, Microscale immobilized protease reactor columns for peptide mapping by liquid chromatography/mass spectral analyses, Anal. Biochem. 224 (1995) 235-244,
Gao, J. Xu, L. E. Locascio, C. S. Lee, Integrated microfluidic system enabling protein digestion, peptide separation, and protein identification, Anal. Chem. 73 (2003) 2648-2655,
Keil-Dlouha, N. Zylber, J. M. Imhoff, N. T. Tong, B. Keil, Proteolytic activity of pseudotrypsin, FEBS Lett. 16 (1971) 291-295,
J. Lippincoft, E. Hess, I. Apostol, Mapping of recombinant hemoglobin using immobilized trypsin cartridges, Analytical Biochem. 252 (1997) 314-325),
N. Lundell, T. Schreitmuller, Sample Preparation for Peptide Mapping—A Pharmaceutical Quality-Control Perspective, Anal. Biochem. 266 (1999) 31-47,
D. S. Peterson, T. Rohr, F. Svec, J. M. J. Fréchet, Enzymatic microreactor-on-a-chip: protein mapping using trypsin immobilized on porous polymer monoliths molded in channels of microfluidic devices, Anal. Chem. 74 (2002) 4081-4088,
D. S. Peterson, T. Rohr, F. Svec, J. M. J. Fréchet, High throughput peptide mass mapping using a micro device containing trypsin immobilized on a porous polymer monolith coupled to MALDI TOF and ESI TOF mass spectrometers, J. Prot. Res. 1 (2002) 563-568,
J. Samskog, D. Bylund, S. P. Jacobsson, K. E. Markides, Miniaturized on-line proteolysis-capillary liquid chromatography-mass spectrometry for peptide mapping of lactate dehydrogenase, J. Chrom. B. 998 (2003) 83-91,
G. W. Slysz, D. C. Schriemer, On-column, digestion of proteins in aqueous-organic solvents, Rapid Commun. Mass. Spectrometry 17 (2003) 1044-1.050,
G. Vecchione, B. Casetta, R. Santacroce, M. Margaglione, A comprehensive on-line digestion-liquid chromatography/mass spectrometry/collision-induced dissociation mass spectrometry approach for the characterization of human fibrinogen, Rapid Commun. Mass. Spectrometry 15 (2001) 1383-1390,
C. Wang, R. Oleschuk, F. Ouchen, J. Li, P. Thibault, D. J. Harrison, Integration of immobilized trypsin bead beds for protein digestion within a microfluidic chip incorporating capillary electrophoresis separations and an electrospray mass spectrometry interface, Rapid Commun. Mass. Spectrometry 14 (2000) 1377-1383.

1.-35. (canceled)

36. An enzyme for protein digestion with at least one amino acid containing an N-terminal amino group and/or an amino group side chain which is modified by a substituent introduced into the enzyme so as to reduce autodigestion and/or enhance protein digestion of the enzyme,
wherein the enzyme is one of:
trypsin, pepsin, lysine-C, glu-C, chymotrypsin, arg-C, asp-N, papain, elastase,
and wherein the enzyme is immobilized on a solid-support material being shaped as one of:
beads, monolith, membrane, or a planar surface.

37. The enzyme of claim 36, wherein one amino group of the at least one amino acid is contained in the side chain, preferably being a tyrosine side chain, of the amino acid.

38. The enzyme of claim 36, wherein the substituent comprises a functional group, preferably one of:
an acetyl,
a methyl,
an alkyl,
a sugar conjugate, the sugar being a monomeric or an oligomeric sugar,
a succinyl,
or a guanidyl group.

39. The enzyme of claim 36, wherein the solid-support-material is one of:
Agarose, in particular Pierce® Agarose Beads,
a silica-based material, in particular porous silica or a silica-based monolith,
a polymethacrylate-based material, in particular a polymethacrylate-based monolith,
polystyrene/divinylbenzene-based material, in particular a polystyrene/divinylbenzene-based monolith,
a Nitrocellulose material, in particular a Nitrocellulose membrane,
Sepharose, in particular an N-hydroxysuccinimide activated Sepharose,
a polystyrene and/or divinylbenzene-based solid-support, in particular a bead shaped polystyrene and/or divinylbenzene-based solid-support,
Dynabeads®.

40. The enzyme of claim 36, wherein the enzyme is immobilized in the presence of a reversible enzyme inhibitor, said enzyme inhibitor preferably being benzamidine.

41. A cartridge for digestion of proteins, wherein the cartridge comprises the enzyme of claim 36 and wherein said cartridge comprises at least one of the following:
slurry-packed solid-support beads on which said enzyme is immobilized,
a monolith which monolith becomes provided with said enzyme, with the modification being performed in the packed cartridge.

42. The cartridge of claim 41 for digestion of proteins, wherein said cartridge is a capillary.

43. A digestion device for digestion of proteins, comprising the enzyme of claim 36, said digestion device having at least one of the features:
reactor size,
microfluidic device size.

44. The digestion device of claim 43, comprising:
a cartridge for digestion of proteins, comprising at least one of the following:
slurry-packed solid-support beads on which said enzyme is immobilized,
a monolith which monolith becomes provided with said enzyme, with the modification being performed in the packed cartridge,
the cartridge holding an enzyme for protein digestion with at least one amino acid containing an N-terminal amino group and/or an amino group side chain which is modified by a substituent introduced into the enzyme so as to reduce autodigestion and/or enhance protein digestion of the enzyme,
wherein the enzyme is one of:
trypsin, pepsin, lysine-C, glu-C, chymotrypsin, arg-C, asp-N, papain, elastase,
and wherein the enzyme is immobilized on a solid-support material being shaped as one of:
beads, monolith, membrane, or a planar surface.

45. An automated protein analysis device, wherein the digestion device of claim 43 is comprised, the automated protein analysis device being adapted for integrated on-line digestion.

46. A method for modification and immobilization of an enzyme, in particular to obtain the enzyme of claim 36, comprising:
bringing in contact of the enzyme with said solid-support, thus performing immobilization of said enzyme,
performing at least one of a chemical, biochemical or physical reaction of said N-terminal amino group or said at least one amino group side chain contained by said at least one amino acid of said enzyme with a modification reagent, whereby reacting of at least one amino group with said modification reagent leads to introduction of an activity and stability enhancing functional group.

47. The method of claim 46, comprising:
preparing said solid-support material,
dissolving a volume of said enzyme at a temperature of −2° C. to 2° C., more preferably at a temperature 0° C., in coupling buffer,
adding said dissolved enzyme to the solid-support,
performing reaction of the enzyme with the solid-support by providing a first incubating and mixing,
removing excess solvent after reaction,
modifying the enzyme by gradually adding of modification reagent,
performing reaction of the enzyme with the modifying reagent by providing a second incubation and mixing,
blocking excess of reactive groups by adding of 1 to 10, preferably 5 volumes of blocking buffer, which preferably comprises 0.1 to 1 M ethanolamine, preferably 0.5 M ethanolamine, at 1 to 100-fold dilutions and pH values ranging 3 to 9,
and providing a third incubating and mixing.

48. The method according to claim 46, wherein the enzymes obtained are stored in storage buffer, said storage buffer comprising preferably least one of the following:
50 mM is tris(hydroxymethyl)aminomethane pH 8.2,
1 mM CaCl₂, 0.02% NaN₃,
wherein the storing temperature ranges from 0° C. to 8° C., preferably it is 4° C.

49. The method of claim 47, wherein preparing of said solid-support material comprises:
washing the solid-support at a temperature of 0° C. to 10° C., more preferably at a temperature of 2° C. to 6° C., most preferably at a temperature of 4° C. with at least 2 volumes of washing buffer.

50. The method of claim 49, wherein the washing buffer comprises at least one of the following:
0.1 to 2 mM HCl, preferably 0.5 to 1.5 mM HCl, most preferably 1 mM HCl,
0.01 to 1 M K₂HPO₄ preferably 0.05 to 0.5 mM K₂HPO₄,
most preferably 0.1 M K₂HPO₄ with pH 7.8.

51. The method according to claim 46, wherein said modification reagent comprises at least one of the following:
a second solvent, in particular one of Acetonitrile, Dimethylsulfoxide, Dimethylformamide, Tetrahydrofuran, Dioxan, Acetone,
an acetylating reagent, in particular acetic acid N-hydroxy-succinimide ester,
an aldehyde, in particular an aldehyde having alkyl chains, in particular alkyl chains ranging from 1 to 12 carbon atoms,
ethylene glycol bis(succinimidyl succinate) to achieve cross linking,
a sugar whose hydroxy-functional groups are converted into aldehydes, in particular a monomeric sugar or an oligomeric sugar such as cyclodextrine,
polyethylene glycol, in particular methoxy-polyethylene glycol, preferably one of N-hydroxy-succinimide activated methoxypolyethylene glycol or p-nitrophenyl chloroformate activated methoxypolyethylene,
a succinyl-group to perform succinylation,
a guanidyl-group to perform guanylation,
chloroanhydrides to perform conjugation,
mixed anhydrides to perform conjugation.

52. The method of claim 47, wherein the coupling buffer comprises at least one of:
a serine protease inhibitor,
0.01 to 1 M K₂HPO₄, preferably 0.05 to 0.5 M K₂HPO₄, most preferably 0.1 M K₂HPO₄ with pH 7.8,
1 to 10 mM ethanolamine, preferably 5 mM ethanolamine,
1 to 10 mM benzamidine, preferably 4 mM benzamidine having pH 7.8.

53. The method of claim 52, comprising:
adding said dissolved enzyme to the solid-support in the presence of the serine protease inhibitor, preferably in the presence of benzamidine.

54. The method of claim 47, comprising at least one of the following:
performing said first incubating and mixing during 25 minutes at a temperature of 25° C. with rotary shaking at 1100 rpm,
performing said second incubating and mixing during 20 minutes at a temperature of 25° C. with rotary shaking at 1100 rpm,
performing said third incubating and mixing is performed during 10 minutes at a temperature of 25° C. with rotary shaking at 1100 rpm.

55. A method of preparing the cartridge of claim 41, comprising:
adding immobilized enzymes comprising solid-supports, in particular beads-shaped solid-supports, to a storage buffer to perform a slurry,
filling said slurry into the cartridge.


SEQUENCE LISTING (9 sequences)

SEQ ID NO: 1 - protein, 7 aa, Artificial Sequence
  Bos taurus trypsin autolysis peptide
KSGIQVR

SEQ ID NO: 2 - protein, 21 aa, Artificial Sequence
  Bos taurus trypsin autolysis peptide
RLGEDNINVVEGNEQFISASK

SEQ ID NO: 3 - protein, 21 aa, Artificial Sequence
  Bos taurus trypsin autolysis peptide
KSIVHPSYNSNTLNNDIMLIK

SEQ ID NO: 4 - protein, 41 aa, Artificial Sequence
  Bos taurus trypsin autolysis peptide
RLGEDNINVVEGNEQFISASKSIVHPSYNSNTLNNDIMLIK

SEQ ID NO: 5 - protein, 11 aa, Artificial Sequence
  Bos taurus trypsin autolysis peptide
KLKSAASLNSR

SEQ ID NO: 6 - protein, 9 aa, Artificial Sequence
  Bos taurus trypsin autolysis peptide
KSAASLNSR

SEQ ID NO: 7 - protein, 12 aa, Artificial Sequence
  Bos taurus trypsin autolysis peptide
KSSGTSYPDVLK

SEQ ID NO: 8 - protein, 22 aa, Artificial Sequence
  Bos taurus trypsin autolysis peptide
KSAYPGQITSNMFCAGYLEGGK

SEQ ID NO: 9 - protein, 7 aa, Artificial Sequence
  Bos taurus trypsin autolysis peptide
KQTIASN